(19) United States
(12) Patent Application Publication
(10) Pub. No.: US 2023/0032944 A1
(43) Pub. Date: Feb. 2, 2023

(54) Ophthalmic Lens With Depth-Modulated Optical Structures and Methods of Forming

(71) Applicants: Clerio Vision, Inc., Rochester, NY (US); University of Rochester, Rochester, NY (US)

(72) Inventors: Lisen J. Xu, Pittsford, NY (US); Brian T. Smith, Fairport, NY (US); Gustavo A. Gandara-Montano, Rochester, NY (US); Wayne H. Knox, Rochester, NY (US)

(73) Assignees: Clerio Vision, Inc., Rochester, NY (US); University of Rochester, Rochester, NY (US)

(21) Appl. No.: 17/957,699

(22) Filed: Sep. 30, 2022

Related U.S. Application Data
(63) Continuation of application No. PCT/US2021/024213, filed on Mar. 25, 2021.
(60) Provisional application No. 63/001,993, filed on Mar. 30, 2020.

(51) Int. Cl. G02C 7/02; G02C 7/04

(52) U.S. Cl. CPC G02C 7/024; G02C 7/04; G02C 2202/12

(57) ABSTRACT

Subsurface optical elements are formed within an ophthalmic lens using modulation of depth to which refractive index change inducing laser pulses are focused within the ophthalmic lens. A system for forming one or more subsurface optical structures within an ophthalmic lens comprises a control unit operatively coupled with a laser pulse source and a focusing assembly. The control unit is configured to control operation of the focusing assembly to sequentially focus each of the sequence of laser pulses onto a respective sub-volume of a sequence of sub-volumes of the ophthalmic lens. The sub-volumes of the sequence of sub-volumes have modulated depths within the ophthalmic lens and varying transverse locations within the ophthalmic lens.

### CROSS REFERENCE TO RELATED APPLICATION DATA

The present application is a Continuation of PCT Appln No. PCT/US2021/024213 filed Mar. 25, 2021; which claims the benefit of U.S. Provisional Appln. No. 63/001,993 filed Mar. 30, 2020; the full disclosures which are incorporated herein by reference in their entirety for all purposes.

### BACKGROUND

Optical aberrations that degrade visual acuity are common. Optical aberrations are imperfections of the eye that degrade focusing of light onto the retina. Common optical aberrations include lower-order aberrations (e.g., astigmatism, positive defocus (myopia) and negative defocus (hyperopia)) and higher-order aberrations (e.g., spherical aberrations, coma and trefoil).

Existing treatment options for correcting optical aberrations include glasses, contact lenses and reshaping of the cornea via laser eye surgery. Additionally, intraocular lenses are often implanted to replace native lenses removed during cataract surgery.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Embodiments described herein are directed to ophthalmic lenses having a subsurface optical structure(s) (e.g., diffractive optical structures and/or non-diffractive optical structures) with depth-modulated refractive index variations and methods of forming. In many embodiments, the depth-modulated refractive index variations are formed via focusing femtosecond duration laser pulses onto a targeted sequence of subsurface volumes of an ophthalmic lens. The depth of focus of the laser pulses is modulated so that depths of the targeted subsurface volumes modulates between an upper limit surface and a lower limit surface offset from the upper limit surface. By modulating the depth of focus of the laser pulses during formation of the subsurface optical structure(s), the resulting subsurface optical structure(s) can be formed with reduced damage induced by the sequence of laser pulses at a given pulse energy level as compared to forming a corresponding subsurface optical structure(s) without modulating the depth of focus of the laser pulses. The reduction in damage induced due to modulating the depth of focus can be used to employ higher pulse energy levels, thereby enabling the formation of a more effective subsurface optical structure(s) due to increased refractive index variation that can be induced by the higher pulse energy levels. The approaches described herein may be useful in forming a subsurface optical structure(s) in any suitable ophthalmic lenses (e.g., intraocular lenses, contact lenses, corneas, glasses, and/or native lenses).

Thus, in one aspect, an ophthalmic lens includes a lens body and a first subsurface optical structure disposed within the lens body. The lens body is made of a transparent material. The first subsurface optical structure includes first sub-volumes of the lens body disposed within a first subsurface layer of the lens body. Each of the first sub-volumes has a respective refractive index different from an adjacent portion of the lens body that does not form part of the first subsurface optical structure. The first sub-volumes are disposed at modulated distances perpendicular to a mid-surface of the first subsurface layer.

The first sub-volumes can have any suitable configuration. For example, each of the first sub-volumes can have a volume less than the maximum volume that can be accommodated within the lens body. Positions of the first sub-volumes can have an amplitude of depth modulation of greater than zero perpendicular to the mid-surface of the first subsurface layer. At least some of the first sub-volumes can be contiguous and form an elongated portion of the first subsurface optical structure. The first subsurface optical structure can have elongated portions. Each of the elongated portions can be separated from each of one or two adjacent of the elongated portions by an intervening line spacing. Each of the elongated portions can include contiguous segments. Some of the contiguous segments can extend through the mid-surface of the first subsurface layer. Each of some of the contiguous segments can be substantially straight, contiguous with an adjacent one of the contiguous segments, and extend in a segment direction that is transverse to an adjoining segment direction in which the adjacent contiguous segment extends.

The ophthalmic lens can be or include any suitable type of ophthalmic lens. For example, the ophthalmic lens can be or include any of a contact lens, an intraocular lens, a lens of a pair of glasses, a cornea and/or a native lens of a person.

The ophthalmic lens can include one or more additional subsurface optical structures. For example, the ophthalmic lens can include a second subsurface optical structure. The second subsurface structure can include second sub-volumes of the lens body disposed within a second subsurface layer of the lens body. Each of the second sub-volumes can have a respective refractive index different from an adjacent portion of the lens body that does not form part of the second subsurface optical structure. The second sub-volumes of the lens body can be disposed at modulated distances perpendicular to a mid-surface of the second subsurface layer. The mid-surface of the second subsurface layer can be separated from the mid-surface of the first subsurface layer by at least 5 microns.

In another aspect, a method of forming one or more sub-surface subsurface optical structures includes forming a first subsurface optical structure within a first subsurface layer of an ophthalmic lens made of a transparent material by inducing changes in refractive index of first sub-volumes of the ophthalmic lens disposed at modulated distances perpendicular to a mid-surface of the first subsurface layer. The method can be used with any suitable ophthalmic lens (e.g., an intraocular lens, a contact lens, a cornea, a native lens, and a lens of a pair of glasses).

In embodiments of the method, the first sub-volumes can have any suitable configuration. For example, each of the first sub-volumes can have a volume less than the maximum volume that can be accommodated within the ophthalmic lens. Positions of some of the first sub-volumes can have an amplitude of at least 5 microns perpendicular to the mid-surface of the first subsurface layer. At least some of the first sub-volumes can be contiguous and form an elongated portion of the first subsurface optical structure. The elongated portion of the first subsurface optical structure can extend at least 5 microns above and below the mid-surface of the first subsurface layer. The first subsurface optical structure can have elongated portions. Each of the elongated portions can be separated from each of one or two adjacent of the elongated portions by an intervening line spacing. Each of the elongated portions can include contiguous segments. Each of some of the contiguous segments can extend through the mid-surface of the first subsurface layer. Each of some of the contiguous segments can be substantially straight, contiguous with an adjacent one of the contiguous segments, and extend in a segment direction that is transverse to an adjoining segment direction in which the adjacent contiguous segment extends.

In embodiments of the method, the ophthalmic lens can be or include any suitable type of ophthalmic lens. For example, the ophthalmic lens can be or include any of a contact lens, an intraocular lens, a lens of a pair of glasses, a cornea and/or a native lens of a person. In some embodiments of the method, the first subsurface optical structure can be formed with the intraocular lens in an implanted state within an eye of a patient.

In many embodiments of the method, the ophthalmic lens can include one or more additional subsurface optical structures. For example, the ophthalmic lens can include a second subsurface optical structure. The second subsurface structure can include second sub-volumes of the lens body disposed within a second subsurface layer of the lens body. Each of the second sub-volumes can have a respective refractive index different from an adjacent portion of the lens body that does not form part of the second subsurface optical structure. The second sub-volumes of the lens body can be disposed at modulated distances perpendicular to a mid-surface of the second subsurface layer. The mid-surface of the second subsurface layer can be separated from the mid-surface of the first subsurface layer by at least 5 microns.

In many embodiments of the method, the first subsurface optical structure can be sequentially formed via a sequence of laser pulses. For example, in many embodiments of the method, the changes in refractive index of sub-volumes of the ophthalmic lens can be induced via a sequence of laser pulses focused at respective subsurface positions within the ophthalmic lens.

In another aspect, a system for forming one or more subsurface sub-surface optical structures within an ophthalmic lens includes a laser pulse source, a focusing assembly and a control unit. The laser pulse source is operable to generate a sequence of laser pulses. Each of the laser pulses is configured to induce a change of refractive index of a sub-volume of an ophthalmic lens when focused onto the sub-volume. The focusing assembly is controllable to focus each respective laser pulse of the sequence of laser pulses onto a respective selected sub-volume of the ophthalmic lens. The respective selected sub-volume can be located at any selected depth of different depths within the ophthalmic lens and can be located at any selected transverse location within the ophthalmic lens in two dimensions. The control unit is operatively coupled with the laser pulse source and the focusing assembly. The control unit is configured to control operation of the focusing assembly to sequentially focus each of the sequence of laser pulses onto a respective sub-volume of a sequence of sub-volumes of the ophthalmic lens. The sub-volumes of the sequence of sub-volumes have modulated depths within the ophthalmic lens and varying transverse locations within the ophthalmic lens. In many embodiments, the system further includes an interface assembly configured to restrain a position and an orientation of an ophthalmic lens relative to the focusing assembly.

For a fuller understanding of the nature and advantages of the present invention, reference should be made to the ensuing detailed description and accompanying drawings.

### BRIEF DESCRIPTION OF THE DRAWINGS

FIG. 1 is a plan view illustration of an ophthalmic lens that includes subsurface optical structures with depth-modulated distribution of refractive index variations, in accordance with embodiments.

FIG. 2 is a plan view illustration of a layer of the subsurface optical structures of the ophthalmic lens of FIG. 1.

FIG. 3 is an X-direction side view illustration of depth-modulated distribution of refractive index variations in the subsurface optical structures of FIG. 1.

FIG. 4 is an Y-direction side view illustration of depth-modulated distribution of refractive index variations in the subsurface optical structures of FIG. 1.

FIG. 5 is a simplified schematic illustration of a laser pulse assembly a system for forming one or more subsurface diffractive optical structures within an ophthalmic lens, in accordance with embodiments.

FIG. 6 is a simplified schematic diagram of some components the system of FIG. 5.

FIG. 7 graphically illustrates diffraction efficiency for near focus and far focus versus phase change height.

FIG. 8 graphically illustrates an example calibration curve for resulting phase change height as a function of laser pulse train optical power.

FIG. 9 graphically illustrates measured first order diffraction efficiency for a first experimental ophthalmic lens that includes subsurface optical structures with depth-modulated distribution of refractive index variations over days following formation of the subsurface optical structures with depth-modulated distribution of refractive index variations.

FIG. 10 graphically illustrates measured added optical power and variation thereof for the first experimental ophthalmic lens over days following formation of the subsurface optical structures with depth-modulated distribution of refractive index variations.

FIG. 11 shows a photograph of the first experimental lens.

FIG. 12 shows a photograph of a first control ophthalmic lens corresponding to the first experimental ophthalmic lens, but lacking z-modulation of the distribution of refractive index variations.

FIG. 13 graphically illustrates measured first order diffraction efficiency for a second experimental ophthalmic lens that includes subsurface optical structures with depth-modulated distribution of refractive index variations and measured first order diffraction efficiency for a second control ophthalmic lens that does not include subsurface optical structures with depth-modulated distribution of refractive index variations over days following formation of the subsurface optical structures.

FIG. 14 graphically illustrates measured added optical power and variation thereof for the second experimental ophthalmic lens and the second control ophthalmic lens over days following formation of the subsurface optical structures.

FIG. 15 shows a photograph of the second experimental ophthalmic lens.

FIG. 16 shows a photograph of the second control ophthalmic lens.

FIG. 17 shows a photograph of a third experimental ophthalmic lens that includes subsurface optical structures with depth-modulated distribution of refractive index variations.

FIG. 18 shows a photograph of a third control ophthalmic lens (corresponding to the third experimental ophthalmic lens) that includes subsurface optical structures without depth-modulated distribution of refractive index variations.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

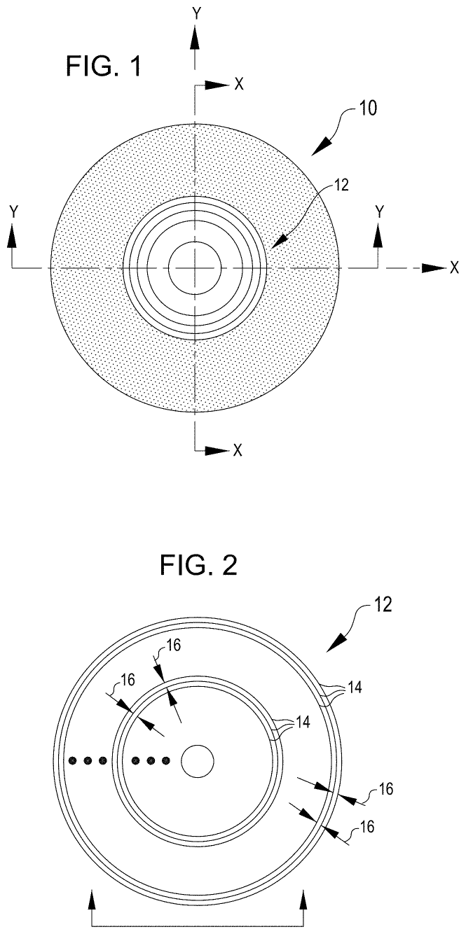

Turning now to the drawing figures, in which like reference numbers refer to like elements in the various figures, FIG. 1 is a plan view illustration of an ophthalmic lens 10 that includes one or more subsurface optical structures 12 with depth-modulated distribution of refractive index variations, in accordance with embodiments. The one or more subsurface structures 12 described herein can be formed in any suitable type of ophthalmic lens including, but not limited to, intra-ocular lenses, contact lenses, corneas, spectacle lenses, and native lenses (e.g., a human native lens). The one or more subsurface optical structures 12 with depth-modulated distribution of refractive index variations can be configured to provide many suitable refractive correction for optical aberrations such as astigmatism, myopia, hyperopia, spherical aberrations, coma and trefoil, as well as any suitable combination thereof.

FIG. 2 is a plan view illustration of one of the subsurface optical structures 12 of the ophthalmic lens 10. The illustrated subsurface optical structure 12 includes concentric circular sub-structures 14 separated by intervening line spaces or gaps 16. In FIG. 2, the size of the intervening line spaces 16 is shown much larger than in many actual embodiments. For example, example embodiments described herein have an outer diameter of the concentric circular sub-structures 14 of 3.75 mm and intervening line spaces 16 of 0.25 um, thereby having 1,875 of the concentric circular sub-structures 14 in embodiments where the concentric circular substructures 14 extend to the center of the subsurface optical structure 12. Each of the concentric circular sub-structures 14 can be formed by focusing suitable laser pulses onto contiguous sub-volumes of the ophthalmic lens 10 so as to induce changes in refractive index of the sub-volumes so that each of the sub-volumes has a respective refractive index different from an adjacent portion of the ophthalmic lens 10 that surrounds the sub-structure 14 and is not part of any of the subsurface optical structures 12.

In many embodiments, a refractive index change is defined for each sub-volume of the ophthalmic lens 10 that form the subsurface optical structures 12 so that the resulting subsurface optical structures 12 would provide a desired optical correction when formed within the ophthalmic lens 10. The defined refractive index changes are then used to determine parameters (e.g., laser pulse power (mW), laser pulse width (fs)) of laser pulses that are focused onto the respective sub-volumes to induce the desired refractive index changes in the sub-volumes of the ophthalmic lens 10.

While the sub-structures 14 of the subsurface optical structures 12 have a circular shape in the illustrated embodiment, the sub-structures 14 can have any suitable shape and distribution and have depth-modulated distribution of refractive index variations. For example, a single sub-structure 14 having an overlapping spiral shape can be employed. In general, one or more substructures 14 having any suitable shapes can be distributed with intervening spaces so as to provide a desired diffraction of light incident on the subsurface optical structure 12.

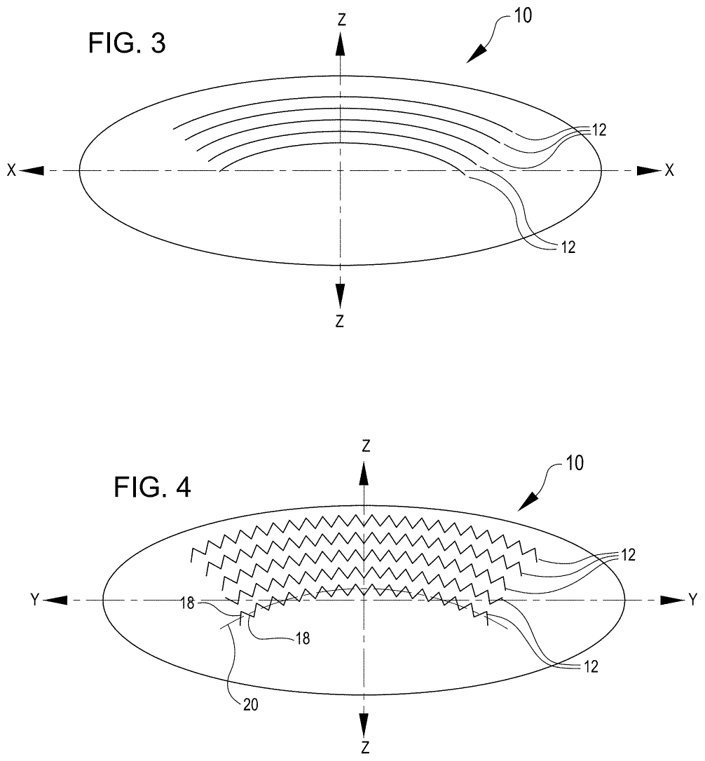

FIG. 3 and FIG. 4 illustrate an embodiment in which the subsurface optical structures 12 are stacked in layers that are separated by intervening layer spaces. In the illustrated embodiment, the subsurface optical structures have a depth-modulated distribution of refractive index variation that are modulated in one direction. FIG. 3 is an X-direction side view illustration of depth-modulated distribution of refractive index variations in the subsurface optical structures 12. FIG. 4 is an Y-direction side view illustration of depth-modulated distribution of refractive index variations in the subsurface optical structures. In the illustrated embodiment, the subsurface optical structures 12 can be formed using a raster scanning approach in which each layer (which comprises one of the subsurface optical structures 12) is sequentially formed starting with the bottom layer and working upward. For each layer, the raster scanning approach sequentially scans the focal position of the laser pulses along planes of constant X-dimension while varying the Y-dimension and the Z-dimension so that the resulting subsurface optical structures 12 have the curved cross-sectional shapes shown in FIG. 3, which shows an X-direction cross-sectional view of the ophthalmic lens 10. The scanned Z-dimensions employed for the scanned planes of constant X-dimension are sequentially modulated via alternating a series of increased Z-dimension with a series of decreased Z-dimension so that the resulting subsurface optical structures 12 have the curved cross-sectional shapes shown in FIG. 4, which shows a Y-direction cross-sectional view of the ophthalmic lens 10. In the raster scanning approach, timing of the laser pulses is controlled to direct each laser pulse onto a targeted sub-volume of the ophthalmic lens 10 and not direct laser pulses onto non-targeted sub-volumes of the ophthalmic lens 10, which include sub-volumes of the ophthalmic lens 10 that do not form any of the subsurface optical structures 12, such as the intervening line spaces 16 and the intervening spaces between adjacent pairs of the subsurface optical structures 12.

The raster scanning approach ensures that adjacent portions of the substructures 14 are formed with a controlled amount of relative Z-direction modulation depth increment, thereby ensuring a suitable relative orientation of adjacent portions of the substructures 14 with respect to orientation of the intervening gap 16 between the adjacent portions of the substructures 14. A suitable relative orientation of the intervening gap 16 between adjacent portions of the substructures 14 may be beneficial in ensuring suitable optical characteristics of the subsurface optical structure 12, including, for example, diffractive characteristics of the subsurface optical structure 12 associated with the orientation of the intervening gap 16.

The Z-direction modulation depth increment can have any suitable configuration. For example, in addition to the one-directionally depth-modulated distribution of refractive index variations described above and illustrated in FIG. 3 and FIG. 4, the depth of the refractive index variations can be concentrically modulated so that the depth is modulated radially from a central point of the subsurface optical structure 12. In such a concentrically depth-modulated distribution, the depth at each radial distance from the central point of the subsurface optical structure 12 can be the same or have any suitable variation based on the desired orientation of the optical structure 12 within the lens 10. In some embodiments, such a concentrically depth-modulated distribution, as illustrated in any radial cross-section (e.g., cross-sections X-X and Y-Y as defined in FIG. 1), can be similar to as shown in FIG. 4.

In the illustrated embodiment, there are five subsurface optical structures 12 with depth-modulated distribution of refractive index variations. Each of the illustrated subsurface optical structures 12 has a generally spherical layer configuration and is separated from each of one or more adjacent of the subsurface optical structures 12 by an intervening layer spacing. Each of the subsurface optical structures 12, however, can alternatively have any other suitable general shape including, but not limited to, any suitable non-planar or planar surface. In the illustrated embodiment, each of the subsurface optical structures 12 has a circular outer perimeter. Each of the subsurface optical structures 12, however, can alternatively have any other suitable outer perimeter shape. Each of the subsurface optical structures 12 can include two or more separate optical substructures with each covering a portion of an overall area of the subsurface optical structures 12.

In the illustrated embodiment, a series of contiguous sub-volumes of the ophthalmic lens 10 form one of the concentric circular sub-structures 14. In some regions of each of the optical structures 12, the contiguous sub-volumes are arranged to form contiguous sloped line segments 18 such as illustrated in FIG. 4. Each of the sloped line segments 18 extend through a mid-surface 20 of the optical structure 12 so that the sub-structure 14 repeatedly and alternately extends above and below the mid-surface 20. The mid-surface 20 can have any suitable shape, for example, spherical, curved in two directions, curved in one direction, or planar. The shape of the mid-surface 20 can be selected based on the exterior shape of the ophthalmic lens 10 and the desired optical correction to be provided by the subsurface optical structure(s) 12. The sloped line segments 18 can extend above the mid-surface 20 by any suitable elevation, and can extend below the mid-surface 20 by any suitable depth. For example, in example embodiments described herein, the elevation and depth of the sloped line segments 18 have are separated by an amplitude of 70 microns. In some regions of each of the optical structures 12, the contiguous sub-volumes are arranged as illustrated in FIG. 3.

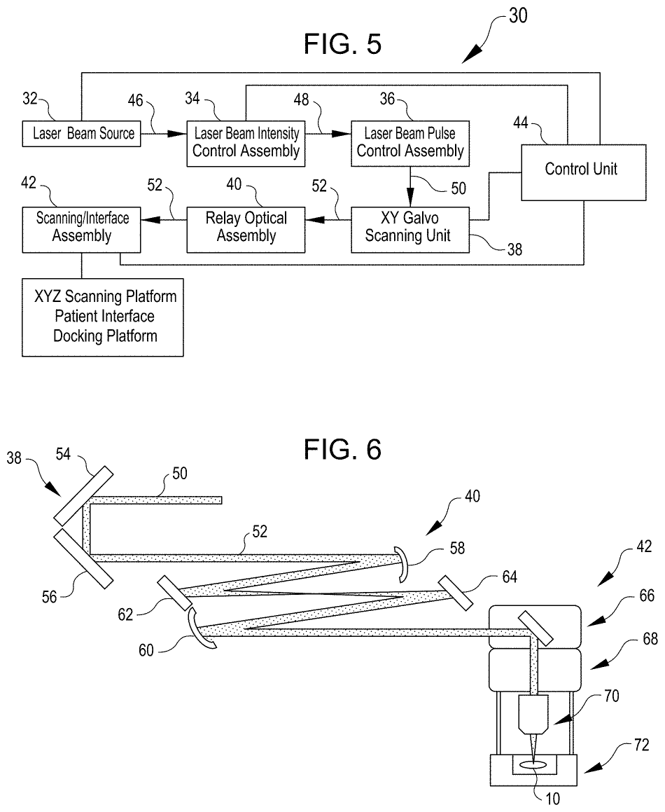

FIG. 5 is a simplified schematic illustration of a system 30 for forming one or more subsurface optical structures 12 within an ophthalmic lens 10, in accordance with embodiments. The system 30 includes a laser beam source 32, a laser beam intensity control assembly 34, a laser beam pulse control assembly 36, an XY galvo scanning unit 38, a relay optical assembly 40, a scanning/interface assembly 42 and a control unit 44.

The laser beam source 32 generates and emits a laser beam 46 having a suitable wavelength for inducing refractive index changes in the target sub-volumes of the ophthalmic lens 10. In examples described herein, the laser beam 46 has a 1035 nm wavelength. The laser beam 46, however, can have any suitable wavelength (e.g., in a range from 400 to 1100 nm) effective in inducing refractive index changes in the target sub-volumes of the ophthalmic lens 10.

The laser beam intensity control assembly 34 is controllable to selectively vary intensity of the laser beam 46 to produce a selected intensity laser beam 48 output to the laser beam pulse control assembly 36. The laser beam intensity control assembly 34 can have any suitable configuration, including any suitable existing configuration, to control the intensity of the resulting laser beam 48.

The laser beam pulse control assembly 36 is controllable to generate collimated laser beam pulses 50 having suitable duration and intensity for inducing refractive index changes in the target sub-volumes of the ophthalmic lens 10. The laser beam pulse control assembly 36 can have any suitable configuration, including any suitable existing configuration, to control the duration of the resulting laser beam pulses 50.

The XY galvo scanning unit 38 is controllable to selectively scan the laser beam pulses 50 to produce XY scanned laser pulses 52. The XY galvo scanning unit 38 can have any suitable configuration, including any suitable existing configuration (for example, the configuration illustrated in FIG. 6) to produce the XY scanned laser pulses 52. In many embodiments, the XY galvo scanning unit 38 is controllable to selectively scan the laser beam pulses 50 in two orthogonal directions transverse to the direction of propagation of the XY scanned laser pulses 52.

The relay optical assembly 40 receives the XY scanned laser pulses 52 from the XY galvo scanning unit 38 and outputs the XY scanned laser pulses 52 to the scanning/interface assembly 42, in a manner that minimizes vignetting. The relay optical assembly 40 can have any suitable configuration to relay the XY scanned laser pulses 52 to the scanning/interface assembly 42. For example, the relay optical assembly 40 can be configured as illustrated in FIG. 6.

The scanning/interface assembly 42 receives the XY scanned laser pulses 52 from the relay optical assembly 40 and is controlled to selectively scan each of the XY scanned laser pulses 52 to generate XYZ scanned laser pulses 54 (see FIG. 6) focused onto targeted sub-volumes of the ophthalmic lens 10 to induce the respective refractive index changes in the targeted sub-volumes so as to form the one or more subsurface optical structures 12 within an ophthalmic lens 10. The scanning/interface assembly 42 can have any suitable configuration, such as the configuration shown in FIG. 6. In many embodiments, the scanning/interface assembly 42 is configured to restrain the position of the ophthalmic lens 10 to a suitable degree to suitably control the location of the targeted sub-volumes of the ophthalmic lens 10 relative to the scanning/interface assembly 42. In many embodiments, such as the embodiment illustrated in FIG. 6, the scanning/interface assembly 42 includes a motorized Z-stage that is controlled to selectively control the depth within the ophthalmic lens 10 to which the laser pulse is focused.

The control unit 44 is operatively coupled with each of the laser beam source 32, the laser beam intensity control assembly 34, the laser beam pulse control assembly 36, the XY galvo scanning unit 38, and the scanning/interface assembly 42. The control unit 44 provides coordinated control of each of the laser beam source 32, the laser beam intensity control assembly 34, the laser beam pulse control assembly 36, the XY galvo scanning unit 38, and the scanning/interface assembly 42 so that each of the XYZ scanned laser pulses 52 have a selected intensity and duration, and are focused onto a respective selected sub-volume of the ophthalmic lens 10 to form the one or more subsurface optical structures 12 within an ophthalmic lens 10. The control unit 44 can have any suitable configuration. For example, in some embodiments, the control unit 44 comprises one or more processors and a tangible memory device storing instructions executable by the one or more processors to cause the control unit 44 to control and coordinate operation of the of the laser beam source 32, the laser beam intensity control assembly 34, the laser beam pulse control assembly 36, the XY galvo scanning unit 38, and the scanning/interface assembly 42 to produce the XYZ scanned laser pulses 52, each of which is synchronized with the spatial position of the sub-volume optical structure.

FIG. 6 is a simplified schematic illustration of embodiments of the XY galvo scanning unit 38, the relay optical assembly 40 and the scanning/interface assembly 42 of the system 30 for forming one or more subsurface optical structures 12 within an ophthalmic lens 10. The XY galvo scanning unit 38 includes XY galvo scan mirrors 54, 56. The relay optical assembly 40 includes concave mirrors 58, 60 and plane mirrors 62, 64. The scanning/interface assembly 42 includes a Z stage 66, an XY stage 68, a focusing objective lens 70, and a patient interface/ophthalmic lens holder 72.

The XY galvo scanning unit 38 receives the laser pulses 50 (e.g., 1035 nm wavelength collimated laser pulses) from the laser beam pulse control assembly 36. In the illustrated embodiment, the XY galvo scanning unit 38 includes a motorized X-direction scan mirror 54 and a motorized Y-direction scan mirror 56. The X-direction scan mirror 54 is controlled to selectively vary orientation of the X-direction scan mirror 54 to vary direction/position of the XY scanned laser pulses 52 in an X-direction transverse to direction of propagation of the XY scanned laser pulses 52. The Y-direction scan mirror 56 is controlled to selectively vary orientation of the Y-direction scan mirror 56 to vary direction/position of the XY scanned laser pulses 52 in an Y-direction transverse to direction of propagation of the XY scanned laser pulses 52. In many embodiments, the Y-direction is substantially perpendicular to the X-direction.

The relay optical assembly 40 receives the XY scanned laser pulses 52 from the XY galvo scanning unit 38 and transfers the laser pulses 52 to the scanning/interface assembly 42, in a manner that minimizes vignetting. Concave mirror 58 reflects each laser pulse 52 to produce a converging laser pulses incident on plane mirror 62. Plane mirror 62 reflects the converging laser pulse towards plane mirror 64. Between the plane mirror 62 and the plane mirror 64, the laser pulse transitions from being convergent to being divergent. The divergent laser pulse is reflected by plane mirror 64 onto concave mirror 60. Concave mirror 60 reflects the laser pulse to produce a collimated laser pulse that is directed to the scanning/interface assembly 42.

The scanning/interface assembly 42 receives the XY scanned laser pulses 52 from the relay optical assembly 40. In the illustrated embodiment, the Z stage 66 and the XY stage 68 are coupled to the focusing objective lens 70 and controlled to selectively position the focusing objective lens 70 relative to the ophthalmic lens 10 for each of the XY scanned laser pulses 52 so as to focus the laser pulse 52 onto a respective targeted sub-volume of the ophthalmic lens 10. The Z stage 66 is controlled to selectively control the depth within the ophthalmic lens 10 to which the laser pulse is focused (i.e., the depth of the sub-surface volume of the ophthalmic lens 10 on which the laser pulse is focused to induce a change in refractive index of the targeted sub-surface volume). The XY stage 68 is controlled in conjunction with control of the XY galvo scanning unit 38 so that the focusing objective lens 70 is suitably positioned for the respective transverse position of each of the XY scanned laser pulses 52 received by the scanning/interface assembly 42. The focusing objective lens 48 converges the laser pulse onto the targeted sub-surface volume. The patient interface/ophthalmic lens holder 72 restrains the ophthalmic lens 10 relative to the scanning/interface assembly 42 to a sufficient degree to support scanning of the laser pulses by the scanning/interface assembly 42 to form the subsurface optical structures 12.

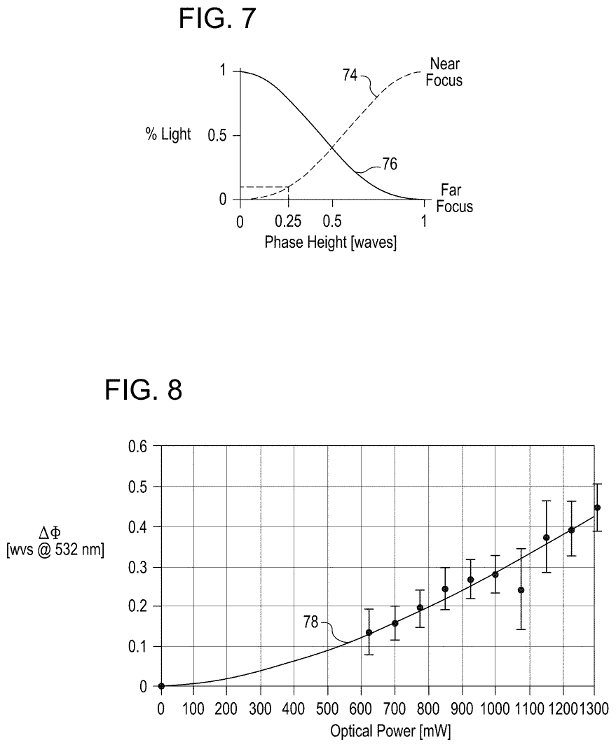

FIG. 7 graphically illustrates diffraction efficiency for near focus 74 and far focus 76 versus phase change height. For phase change heights less than 0.25 waves, the diffraction efficiency for near focus is only about 10 percent. Near focus diffraction efficiency of substantially greater than 10 percent, however, is desirable to limit the number of the subsurface optical structures 12 that are stacked to generate a desired overall optical correction. Greater phase change heights can be achieved by inducing greater refractive index changes in the targeted sub-volumes of the ophthalmic lens 10. Greater refractive index changes in the targeted sub-volumes of the ophthalmic lens 10 can be induced by increasing energy of the laser pulses focused onto the targeted sub-volumes of the ophthalmic lens 10.

FIG. 8 graphically illustrates an example calibration curve 78 for resulting phase change height as a function of laser pulse optical power. The calibration curve 78 shows correspondence between resulting phase change height as a function of laser average power for a corresponding laser pulse duration, laser pulse wavelength, laser pulse repetition rate, numerical aperture, material of the ophthalmic lens 10, depth of the targeted sub-volume, spacing between the targeted sub-volumes, scanning speed, and line spacing between the sub-structures 14. The calibration curve 78 shows that increasing laser pulse energy results in increased phase change height.

Laser pulse energy, however, may be limited to avoid propagation of damage induced caused by laser pulse energy and/or heat accumulation with the ophthalmic lens 10 along and across the sub-structures 14, or even between the layered subsurface optical structures 12. In many instances, there is no observed damage during formation of the first two of layered subsurface optical structures 12 and damage starts to occur during formation of the third of layered subsurface optical structures 12. To avoid such damage, the formation of layered subsurface optical structures 12 can be accomplished using laser pulse energy far below a pulse energy threshold of the material of the ophthalmic lens 10. Using lower pulse energy, however, increases the number of the subsurface optical structures 12 that must be layered to provide the same amount of resulting phase change height, thereby adding to the time required to form the total number of subsurface optical structures 12 employed. For in-vivo applications (for example, writing the subsurface optical structures 12 into an implanted intra-ocular lens), forming additional layered subsurface optical structures 12 may potentially increase an overall amount of energy that is deposited into the retina.

Experimental Depth-Modulated Refractive Index Structures

Experimental depth-modulated refractive index structures were created using continuous reciprocating motion of the Z-stage 66 during formation of layered subsurface optical structures 12. Corresponding control refractive index structures were created without using continuous reciprocating motion of the Z-stage 66 during formation of layered subsurface optical structures 12 for comparison with the depth-modulated refractive index structures. The experimental depth-modulated refractive index structures were created using an amplitude of modulation of the Z-stage 66 of 70 microns and frequency of modulation of 75 oscillations per minute.

It was discovered that the depth-modulated refractive index structures exhibited improved attributes relative to the corresponding control refractive index structures. The depth-modulated refractive index structures exhibited less laser pulse energy induced damage (e.g., fewer and smaller burned portions of the corresponding ophthalmic lens 10) as compared to the corresponding control refractive index structures. The depth-modulated refractive index structures exhibited less laser pulse energy induced damage propagation as compared to the corresponding control refractive index structures. The depth-modulated refractive index structures also exhibited substantially equivalent diffractive efficiency as compared to the corresponding control refractive index structures. In view of the reduced laser pulse energy induced damage obtained using continuous reciprocating motion of the Z-stage 66 during formation of layered subsurface optical structures 12 as compared to not using continuous reciprocating motion of the Z-stage 66, it is believed that the use of continuous reciprocating motion of the Z-stage 66 during formation of layered subsurface optical structures 12 can be used to enable the use of increased laser pulse energy, thereby enabling greater induced refractive index changes, which provides a corresponding increase in diffraction efficiency of corresponding layered subsurface optical structures 12. In many embodiments, an ophthalmic lens 10 has sufficient depth to accommodate an increase in overall height of layered subsurface optical structures 12 formed therein to accommodate the increased thickness of each optical structure 12 caused by the Z modulation. Continuous reciprocating motion of the Z-stage 66 during formation of layered subsurface optical structures 12 can be used to increase the achievable diffraction efficiency of near focus for an ophthalmic lens 10 of a given material, simply due to being able to induce increased refractive index change through the use of higher laser pulse energy without causing unacceptable laser pulse energy induced damage.

TABLE 1
Experimental Parameters

| Parameters | Value or Range | |
|---|---|---|
| Laser wavelength | 1035 | nm |
| Laser pulse repetition rate | 5-15 | MHz |
| Laser pulse width | 50-250 | fs (femtosecond) |
| Numerical aperture (NA) | 0.15-0.50 | |
| Scan speed | 250-2000 | mm/sec. |
| Line spacing | 0.25-1.00 | um (micron) |
| Number of layers | 1-12 | |
| Layer spacing | 5-100 | um (micron) |
| Laser average power | 500-2000 | mW |
| Writing depth from apex | 100-750 | um (micron) |
| Subsurface optical structure diameter | 3-6 | mm |
| Optical structure ADD power | 2-4 | (diopters) |

First Experiment

Two ophthalmic lenses were written in the first experiment. Layered subsurface diffractive optical structures consisting of five diffractive structures were formed in each of the two lenses. Each of the diffractive structures had a spherical layer configuration. Each of the two lenses were formed using the same laser pulse energies and the same layer spacing. One of the two lenses (“E1” for experimental lens 1) was formed using the raster scanning approach described herein (which employs reciprocating motion of the Z-stage 66) during formation of layered sub-surface diffractive optical structures 12. The other of the two lenses (“C1” for control lens 1) was formed without using continuous reciprocating motion of the Z-stage 66 during formation of layered sub-surface diffractive optical structures 12. Table 1 shows specifications applicable the experiments described herein.

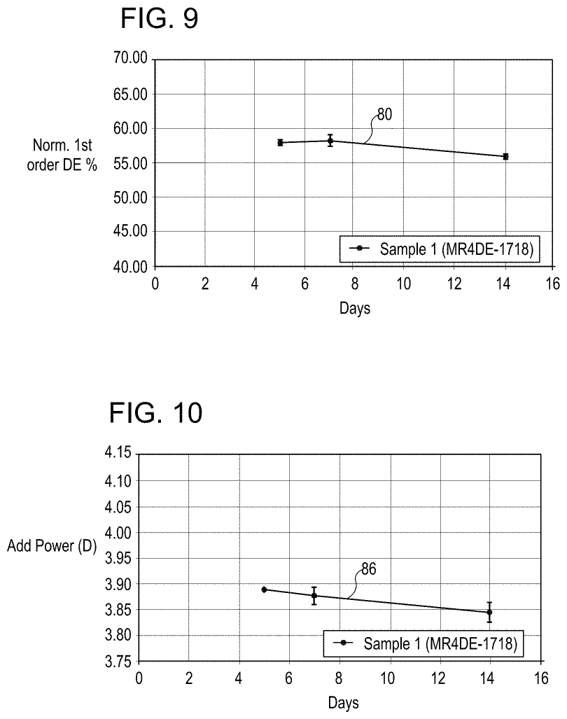

FIG. 9 graphically illustrates measured first order diffraction efficiency 80 for lens E1 over days following formation of the sub-surface diffractive optical structures with depth-modulated distribution of refractive index variations. The measured first order diffraction efficiency 80 of lens E1 averaged 57 percent.

FIG. 10 graphically illustrates measured added optical power and variation thereof for lens E1 over days following formation of the sub-surface diffractive optical structures with depth-modulated distribution of refractive index variations. The design add power of the first experimental lens configuration was 4.0 D. The average measured add power 86 of lens E1 was 3.88 D.

Diffraction efficiency and add power were measured using a Badal Optometer. The measurements were taken in oil. Three measurements were taken of each of lens E1 and lens C1 at each time point. All measured data was averaged.

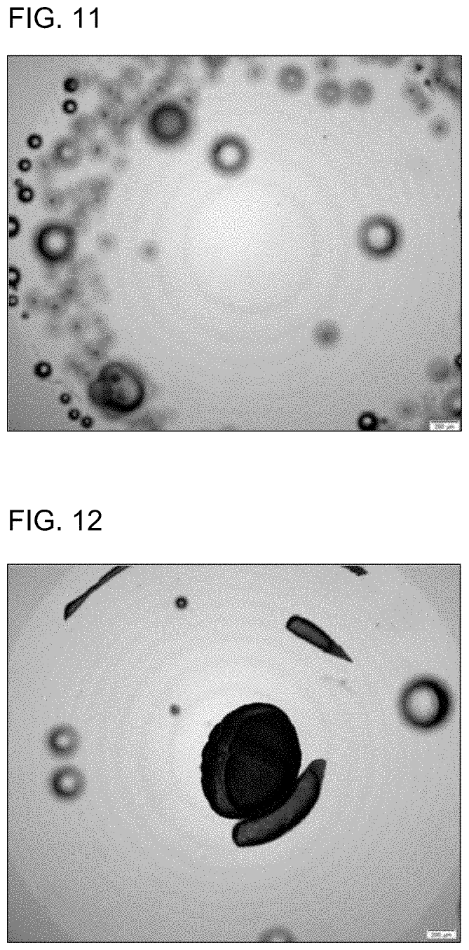

FIG. 11 shows a photograph of lens E1. Lens E1 did not exhibit any burning. The bubbling shown in FIG. 11 is believed to be the result of focusing the laser pulses onto sub-volumes too close to the surface of lens E1.

FIG. 12 shows a photograph of lens C1. Lens C1 burned in five different areas. Even though sub-surface diffractive optical structures were formed in both lens E1 and lens C1 using laser pulse energies below a damage threshold for the MR4DE material forming both lens E1 and lens C1, lens C1 was burned to extent that diffraction efficiency and add power could not be measured for lens C1.

Second Experiment

Two ophthalmic lenses were written in the second experiment. Layered sub-surface diffractive optical structures consisting of five diffractive structures were formed in each of the two lenses. Each of the diffractive structures had a spherical layer configuration. Each of the two lenses were formed using the same laser pulse energies and the same layer spacing between adjacent of the diffractive structures. One of the two lenses (“E2” for experimental lens 2) was formed using the raster scanning approach described herein during formation of layered sub-surface diffractive optical structures 12. The other of the two lenses (“C2” for control lens 2) was formed without using continuous reciprocating motion of the Z-stage 66 during formation of layered sub-surface diffractive optical structures 12. The two lenses in the second experiment were written using 75 mW less laser pulse power than those written in the first experiment in order to obtain measurable results for lens C2.

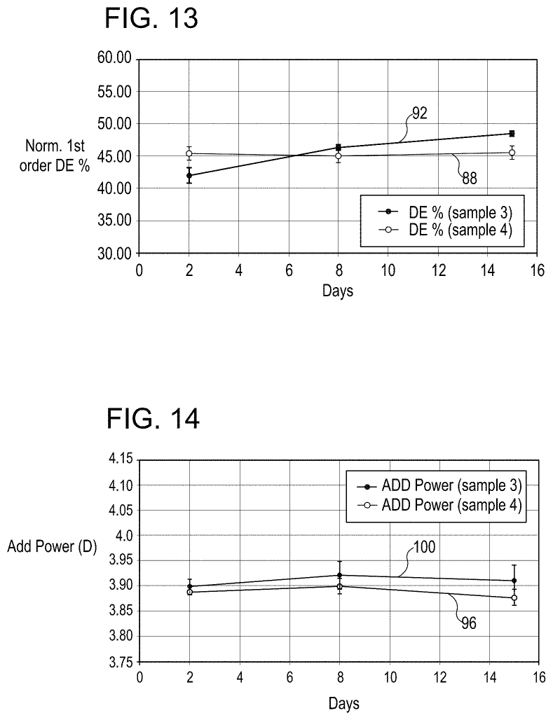

FIG. 13 graphically illustrates measured first order diffraction efficiency 88 for lens E2 over days following formation of the sub-surface diffractive optical structures with depth-modulated distribution of refractive index variations. The measured first order diffraction efficiency 88 of lens E2 averaged 45 percent.

FIG. 13 also graphically illustrates measured first order diffraction efficiency 92 for lens C2 over days following formation of the sub-surface diffractive optical structures without depth-modulated distribution of refractive index variations. The measured first order diffraction efficiency 92 of lens C2 averaged 45 percent—substantially the same as for lens E1.

FIG. 14 graphically illustrates measured added optical power 96 and variation thereof for lens E2 over days following formation of the sub-surface diffractive optical structures with depth-modulated distribution of refractive index variations. The design add power of lens E2 was 4.0 D. The average measured add power 96 of lens E2 was 3.85 D.

FIG. 14 also graphically illustrates measured added optical power 100 and variation thereof for lens C2 over days following formation of the sub-surface diffractive optical structures without depth-modulated distribution of refractive index variations. The design add power of lens C2 was 4.0 D. The average measured add power 100 of lens C2 was 3.9 D—substantially the same as for lens E2.

Diffraction efficiency and add power were measured using a Badal Optometer. The measurements were taken in oil. Three measurements were taken of each of lens E2 and lens C2 at each time point. All measured data was averaged.

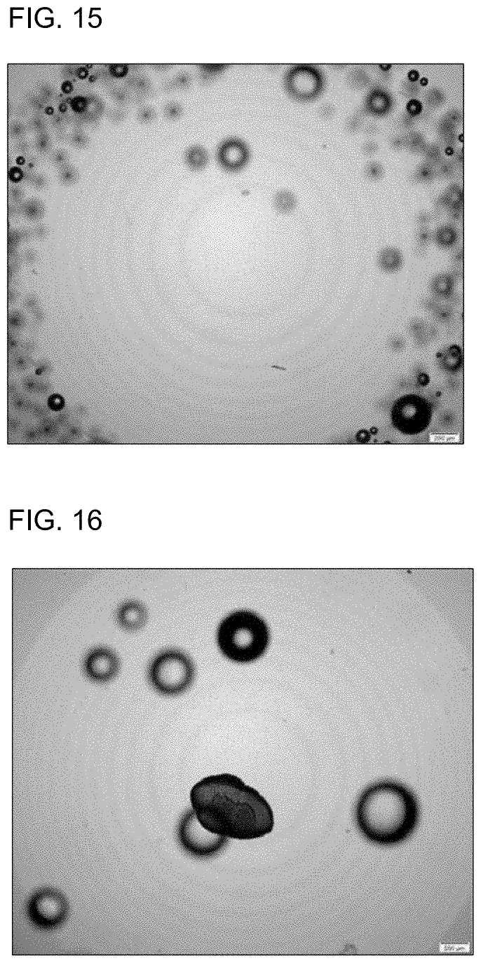

FIG. 15 shows a photograph of lens E2. Lens E2 did exhibit a burned area that did not propagate after igniting. The bubbling shown in lens E2 is believed to be the result of focusing the laser pulses onto sub-volumes too close to the surface of lens E2 due to the Z modulation.

FIG. 16 shows a photograph of lens C2. The burned area seen in lens C2 propagated and spread after igniting.

Third Experiment

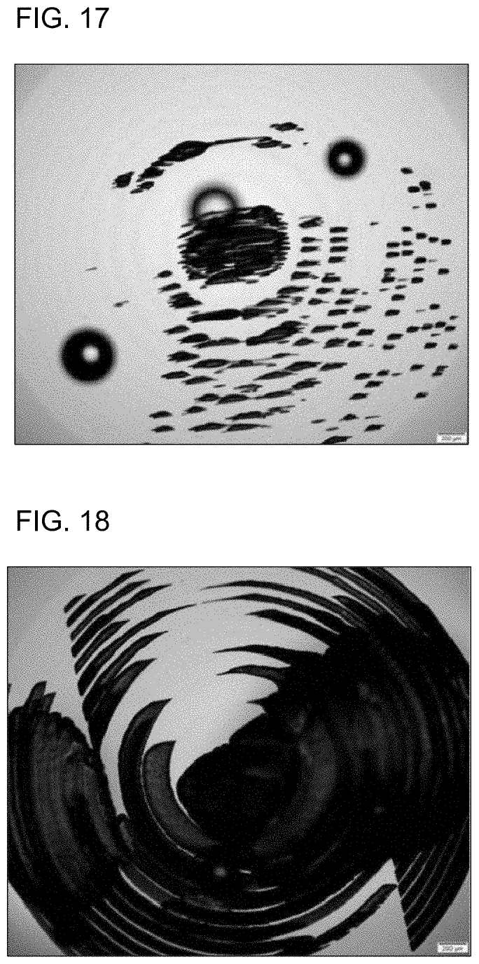

Two ophthalmic lenses were written in the third experiment. Layered sub-surface diffractive optical structures consisting of five diffractive structures were formed in each of the two lenses. Each of the diffractive structures had a spherical layer configuration. Each of the two lenses were formed using the same high laser pulse energies and the same layer spacing between adjacent of the planar diffractive structures. One of the two lenses (“E3” for experimental lens 3) was formed using the raster scanning approach described herein during formation of layered sub-surface diffractive optical structures 12. The other of the two lenses (“C3” for control lens 3) was formed without using continuous reciprocating motion of the Z-stage 66 during formation of layered sub-surface diffractive optical structures 12. The two lenses in the third experiment were written using the same high laser pulse energies selected to ensure burning in both lens E3 and lens C3. FIG. 17 shows a photograph of lens E3. FIG. 18 shows a photograph of lens C3. The greater extent of burned areas in the lens C3 as compared to lens E3 clearly shows the impact of writing with Z-modulation on reducing burning. Laser induced bubbling is not seen in either of lens E3 or lens C3, thereby demonstrating that when the subsurface diffractive structures are written sufficiently deep within the lens, that excessive bubbling near the lens surface is avoided.

Non-Limiting Example Embodiments are Set Forth Below

Example 1 is an ophthalmic lens that includes a lens body made of a transparent material and a first subsurface optical structure that includes first sub-volumes of the lens body disposed within a first subsurface layer of the lens body, wherein each of the first sub-volumes has a respective refractive index different from an adjacent portion of the lens body that does not form part of the first subsurface optical structure, and wherein the first sub-volumes are disposed at modulated distances perpendicular to a mid-surface of the first subsurface layer. Example 2 is an ophthalmic lens in accordance with example 1, wherein positions of a subset of the first sub-volumes have an amplitude of at least 5 microns perpendicular to the mid-surface of the first subsurface layer. Example 3 is an ophthalmic lens in accordance with example 2, wherein at least some of the first sub-volumes are contiguous and form an elongated portion of the first subsurface optical structure. Example 4 is an ophthalmic lens in accordance with example 3, wherein portions of the first subsurface optical structure extend at least 20 microns above and below the mid-surface of the first subsurface layer. Example 5 is an ophthalmic lens in accordance with example 4, wherein the first subsurface optical structure includes elongated portions and each of the elongated portions is separated from each of one or two adjacent of the elongated portions by an intervening line spacing. Example 6 is an ophthalmic lens in accordance with example 5, wherein each of the elongated portions includes contiguous segments and each of the contiguous segments extends through the mid-surface of the first subsurface layer. Example 7 is an ophthalmic lens in accordance with example 6, wherein each of the contiguous segments is substantially straight, is contiguous with an adjacent one of the contiguous segments, and extends in a segment direction that is transverse to an adjoining segment direction in which the adjacent contiguous segment extends.

Example 8 is an ophthalmic lens in accordance with any one of examples 1-7, wherein the ophthalmic lens includes a contact lens. Example 9 is an ophthalmic lens of any one of examples 1-7, wherein the ophthalmic lens includes an intraocular lens. Example 10 is an ophthalmic lens of any one of examples 1-7, further including a second subsurface optical structure including second sub-volumes of the lens body disposed within a second subsurface layer of the lens body, wherein each of the second sub-volumes has a respective refractive index different from an adjacent portion of the lens body that does not form part of the second subsurface optical structure, and wherein the second sub-volumes of the lens body are disposed at modulated distances perpendicular to a mid-surface of the second subsurface layer. Example 11 is an ophthalmic lens in accordance with example 10, wherein the mid-surface of the second subsurface layer is separated from the mid-surface of the first subsurface layer by at least 5 microns.

Example 12 is an ophthalmic lens in accordance with any one of examples 1-7, wherein the first sub-volumes are disposed at distances perpendicular to the mid-surface of the first subsurface layer that have a one-directionally modulated depth distribution. Example 13 is an ophthalmic lens in accordance with any one of examples 1-7, wherein the first sub-volumes are disposed at distances perpendicular to the mid-surface of the first subsurface layer that have a concentrically modulated depth distribution.

Example 14 is a method of forming one or more subsurface optical structures, the method including forming a first subsurface optical structure within a first subsurface layer of an ophthalmic lens made of a transparent material by inducing changes in refractive index of first sub-volumes of the ophthalmic lens disposed at modulated distances perpendicular to a mid-surface of the first subsurface layer. Example 15 is a method in accordance with example 14, wherein positions of a subset of the first sub-volumes have an amplitude of at least 5 microns perpendicular to the mid-surface of the first subsurface layer. Example 16 is a method in accordance with example 15, wherein at least some of the first sub-volumes are contiguous and form an elongated portion of the first subsurface optical structure. Example 17 is a method in accordance with example 16, wherein portions of the elongated portion of the first subsurface optical structure extend at least 20 microns above and below the mid-surface of the first subsurface layer. Example 18 is a method in accordance with example 17, wherein the first subsurface optical structure includes elongated portions and each of the elongated portions is separated from each of one or two adjacent of the elongated portions by an intervening line spacing. Example 19 is a method in accordance with example 18, wherein each of the elongated portion includes contiguous sub-portions.

Example 20 is a method in accordance with any one of examples 14-18, wherein the ophthalmic lens includes a contact lens. Example 21 is a method in accordance with any one of examples 14-18, wherein the ophthalmic lens includes an intraocular lens. Example 22 is a method in accordance with example 21, wherein the first subsurface optical structure is formed with the intraocular lens in an implanted state within an eye of a patient. Example 23 is a method in accordance with any one of examples 14-18, further including forming a second subsurface optical structure within a second subsurface layer of the ophthalmic lens by inducing changes in refractive index of second sub-volumes of the ophthalmic lens disposed at modulated distances perpendicular to mid-surface of the second subsurface layer. Example 24 is a method in accordance with example 23, wherein the mid-surface of the second subsurface layer is separated from the mid-surface of the first subsurface layer by at least 5 micros. Example 25 is a method in accordance with any one of examples 14-18, wherein the changes in refractive index of sub-volumes of the ophthalmic lens are induced via a sequence of laser pulses focused at respective subsurface positions within the ophthalmic lens. Example 26 is a method in accordance with any one of examples 14-18, wherein the first sub-volumes are disposed at distances perpendicular to the mid-surface of the first subsurface layer that have a one-directionally modulated depth distribution. Example 27 is a method in accordance with any one of examples 14-18, wherein the first sub-volumes are disposed at distances perpendicular to the mid-surface of the first subsurface layer that have a concentrically modulated depth distribution.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. An ophthalmic lens comprising:
a lens body made of a transparent material having a lens material refractive index, wherein the lens body comprises first sub-volumes of the lens body having a first distribution of refractive index variations relative to the lens material refractive index, wherein the first sub-volumes of the lens body form a first optical structure configured to provide a first refractive correction, wherein the first optical structure is disposed within a first layer of the lens body and comprises a first elongated portion formed of a first contiguous sequence of the first sub-volumes disposed at depth modulated distances perpendicular to a mid-surface of the first layer so that the first elongated portion of the first optical structure comprises segments that extend through the mid-surface of the first layer.

2. The ophthalmic lens of claim 1, wherein the segments of the first elongated portion extend at least 5 microns above and below the mid-surface of the first subsurface layer of the lens body.

3. (canceled)

4. The ophthalmic lens of claim 2, wherein the segments of the first elongated portion of the first optical structure extend at least 20 microns above and below the mid-surface of the first layer of the lens body.

5. The ophthalmic lens of claim 4, wherein:
the first optical structure comprises a second elongated portion formed of a second contiguous sequence of the first sub-volumes disposed at depth modulated distances perpendicular to a mid-surface of the first layer so that the second elongated portion comprises segments that extend through the mid-surface of the first layer; and
the second elongated portion is separated from the first elongated portion by an intervening line spacing.

6. (canceled)

7. The ophthalmic lens of claim 4, wherein each of the segments of the first elongated portion is substantially straight and extends transverse to an adjoining segment of the segments of the first elongated portion extends.

8. The ophthalmic lens of claim 1, wherein the ophthalmic lens comprises a contact lens.

9. The ophthalmic lens of claim 1, wherein the ophthalmic lens comprises an intraocular lens.

10. The ophthalmic lens of claim 1, wherein the lens body further comprises second sub-volumes of the lens body having a second distribution of refractive index variations relative to the lens material refractive index, wherein second sub-volumes of the lens body form a second optical structure configured to provide a second refractive correction, wherein the second optical structure is disposed within a second layer of the lens body and comprises a first elongated portion formed of a contiguous sequence of the second sub-volumes of the lens body disposed at depth modulated distances perpendicular to a mid-surface of the second layer so that the first elongated portion of the second optical structure comprises segments that extend through the mid-surface of the second layer.

11. The ophthalmic lens of claim 10, wherein the mid-surface of the second layer is separated from the mid-surface of the first layer by at least 5 microns.

12. The ophthalmic lens of claim 1, wherein the first sub-volumes that form the first elongated portion of the first optical structure are disposed at distances perpendicular to the mid-surface of the first layer that have a one-directionally modulated depth distribution.

13. The ophthalmic lens of claim 1, wherein the first sub-volumes that form the first elongated portion of the first optical structure are disposed at distances perpendicular to the mid-surface of the first layer that have a concentrically modulated depth distribution.

14. A method of inducing a distribution of refractive index variations within an ophthalmic lens, the method comprising:
focusing a first sequence of laser pulses onto a sequence of first sub-volumes of a lens body to induce changes in refractive indexes of the sequence of first sub-volumes to form a first optical structure within the lens body that provides a first refractive correction, wherein the first sequence of laser pulses is scanned and a depth of focus of the first sequence of laser pulses is modulated so that the first optical structure is disposed within a first layer of the lens body and comprises an elongated portion formed of a contiguous sequence of the sequence of first sub-volumes disposed at depth modulated distances perpendicular to a mid-surface of the first layer so that the elongated portion of the first optical structure comprises segments that extend through the mid-surface of the first layer.

15. The method of claim 14, wherein the first sequence of laser pulses is scanned and the depth of focus of the first sequence of laser pulses is modulated so that the segments of the elongated portion of the first optical structure extend at least 5 microns above and below the mid-surface of the first layer of the lens body.

16. (canceled)

17. The method of claim 15, wherein the segments of the elongated portion of the first optical structure extend at least 20 microns above and below the mid-surface of the first layer of the lens body.

18. The method of claim 17, wherein: further comprising:
focusing a second sequence of laser pulses onto a sequence of second sub-volumes of the lens body to induce changes in refractive indexes of the sequence of second sub-volumes to form a second optical structure within the lens body that provides a second refractive correction, wherein the second sequence of laser pulses is scanned and a depth of focus of the second sequence of laser pulses is modulated so that the second optical structure is disposed within a second layer of the lens body and comprises an elongated portion formed of a contiguous sequence of the sequence of second sub-volumes disposed at depth modulated distances perpendicular to a mid-surface of the second layer so that the elongated portion of the second optical structure comprises segments that extend through the mid-surface of the second layer.

19. (canceled)

20. The method of claim 14, wherein the ophthalmic lens comprises a contact lens.

21. The method of claim 14, wherein the ophthalmic lens comprises an intraocular lens.

22. The method of claim 21, wherein the first optical structure is formed with the intraocular lens in an implanted state within an eye of a patient.

23. (canceled)

24. The method of claim 18, wherein the mid-surface of the second layer is separated from the mid-surface of the first layer by at least 5 micros.

25. (canceled)

26. The method of claim 14, wherein the sequence of first sub-volumes are disposed at distances perpendicular to the mid-surface of the first layer that have a one-directionally modulated depth distribution.

27. The method of claim 14, wherein the sequence of first sub-volumes are disposed at distances perpendicular to the mid-surface of the first layer that have a concentrically modulated depth distribution.

28. A system for inducing a distribution of refractive index variations within an ophthalmic lens, the system comprising:
a laser pulse source operable to generate a sequence of laser pulses, each of the sequence of laser pulses being configured to induce a change of refractive index of a sub-volume of a lens body of an ophthalmic lens when focused onto the sub-volume;
a focusing assembly controllable to focus each respective laser pulse of the sequence of laser pulses onto a respective selected sub-volume of the ophthalmic lens, wherein the respective selected sub-volume can be located at any selected depth of different depths within the ophthalmic lens and can be located at any selected transverse location within the ophthalmic lens in two dimensions; and
a control unit operatively coupled with the laser pulse source and the focusing assembly, wherein the control unit is configured to control operation of the focusing assembly to sequentially focus each of the sequence of laser pulses onto a respective sub-volume of a sequence of sub-volumes of the ophthalmic lens to form an optical structure within the ophthalmic lens, wherein the sequence of laser pulses is scanned and a depth of focus of the sequence of laser pulses is modulated so that the optical structure is disposed within a first layer of the lens body and comprises an elongated portion formed of a contiguous sequence of the selected sub-volumes disposed at depth modulated distances perpendicular to a mid-surface of the first layer so that the elongated portion of the optical structure comprises segments that extend through the mid-surface of the first layer.

29. The system of claim 28, further comprising an interface assembly configured to restrain a position and an orientation of an ophthalmic lens relative to the focusing assembly.

30. The system of claim 28, wherein the ophthalmic lens comprises a contact lens.

31. The system of claim 28, wherein the ophthalmic lens comprises an intraocular lens.

32. The system of claim 31, configured to form the optical structure with the intraocular lens in an implanted state within an eye of a patient.
